(19) United States
(12) Patent Application Publication
(10) Pub. No.: US 2025/0195597 A1
(43) Pub. Date: Jun. 19, 2025

(54) GUMMY DOSAGE FORMS

(71) Applicant: Zarbee's, Inc., Summit, NJ (US)

(72) Inventors: Amanda Rogers, American Fork, UT (US); Cameron Mylroie, Pleasant Grove, UT (US)

(21) Appl. No.: 18/577,617

(22) PCT Filed: Jul. 7, 2022
(86) PCT No.: PCT/IB2022/056302

§ 371 Date: Jan. 8, 2024

Related U.S. Application Data
(60) Provisional application No. 63/219,505, filed on Jul. 8, 2021.

(51) Int. Cl. A61K 36/185; A23G 3/36; A23G 3/42; A23G 3/48; A23G 3/54; A61K 9/00; A61K 31/375; A61K 35/644; A61K 36/53; A61K 47/12; A61K 47/26; A61K 47/36

(52) U.S. Cl. CPC A61K 36/577; A23G 3/368; A23G 3/42; A23G 3/48; A23G 3/545; A61K 9/0056; A61K 31/375; A61K 35/644; A61K 36/185; A61K 36/53; A61K 47/12; A61K 47/26; A61K 47/36

(57) ABSTRACT

Gummy dosage forms and methods of making and using the same are provided. Such dosage forms can have multiple components including center and outer portions in which at least one portion comprises one or more therapeutic ingredients. The center portion can be a liquid or semi-solid and have a different texture than the outer portion. The outer portion can be a semi-solid or solid, at least partially cover the center portion, and include marshmallow root.

### CROSS-REFERENCE TO RELATED APPLICATIONS

This application claims the benefit of priority to U.S. Provisional Patent Appln. Ser. No. 63/219,505, filed on Jul. 8, 2021, the contents of which are incorporated herein by reference in their entirety.

### 1. FIELD

The present disclosure relates to gummy dosage forms including at least a liquid or semi-solid center portion and an outer gummy shell portion in which at least one portion includes one or more therapeutic ingredients, and processes of manufacturing the same. The present disclosure further relates to a method of treating a subject suffering from an upper respiratory condition, for example, cough, using such gummy dosage forms.

### 2. BACKGROUND

Gummy dosage forms are known for their convenience and versatility in delivering therapeutic ingredients in the medicinal, nutritional, and natural space. They have been utilized to deliver a wide variety of ingredients such as pain treatment ingredients, digestive health treatment ingredients, cough-cold treatment ingredients, nutritional supplements, and natural or herbal ingredients. In certain cases, gummy dosage forms are known as an alternative delivery format for consumers who cannot easily swallow pills, capsules, or tablets, or for consumers who do not prefer medicinal liquids.

In certain gummy dosage forms, there exists a need to have more than one component or for different portions of the dosage form to have different textures. In certain aspects, there also exists a need to have ingredients separated into different portions of the dosage form, for example, to reduce or prevent interaction of various ingredients with each other. More specifically, gummy dosage forms can include a liquid or semi-solid center portion filled within an outer gummy shell portion.

One of the natural ingredients that can be administered to a consumer in a gummy dosage form is marshmallow root. Marshmallow root, also known as Althaea officinalis, is indicated as a natural and herbal ingredient for the treatment of upper respiratory conditions including cough. However, incorporation of marshmallow root in a liquid form or in a liquid center of a gummy dosage form presents a challenge as it adversely affects the viscosity of the liquid or liquid center, requiring separation and incorporation into a different portion of the dosage form. In particular, certain botanicals such as marshmallow root include polysaccharides (i.e., mucilage) that can become gummy when mixed with water.

Accordingly, as there has been ongoing consumer demand for more natural and herbal ingredients in the treatment of, for example, upper respiratory conditions such as cough, there remains a need in the art for the incorporation of natural and herbal ingredients such as marshmallow root into gummy dosage forms. The presently disclosed subject matter addresses these and other needs.

### 3. SUMMARY

The presently disclosed subject matter provides improved gummy dosage forms which can include natural and herbal ingredients, such as marshmallow root. Such gummy dosage forms include a liquid or semi-solid center portion at least partially surrounded by an outer gummy shell portion, in which the outer gummy shell portion includes marshmallow root. The presently disclosed dosage format advantageously ensures the texture of a liquid or semi-solid center fill while retaining a firmer outer gummy shell portion, providing consumers with a convenient and pleasant dosage form for the treatment of upper respiratory conditions such as cough.

The present disclosure provides a gummy form. The gummy form includes a shell portion and a center fill portion. The shell portion includes marshmallow root.

In certain embodiments, the shell portion can wholly encompass the center fill portion.

In certain embodiments, the center fill portion can include a liquid.

In certain embodiments, the center fill portion can include honey.

In certain embodiments, the center fill portion can include honey in an amount of from about 1 mL to about 4 mL. In certain embodiments, the center fill portion can include honey in an amount of from about 2 mL to about 3 mL. In certain embodiments, the center fill portion can include honey in an amount of about 2.5 mL.

In certain embodiments, the center fill portion can be substantially free of marshmallow root.

In certain embodiments, the shell portion can include at least 51% of the total amount of marshmallow root.

In certain embodiments, the gummy form can be substantially free of one or more of high fructose corn syrup, artificial sweeteners, artificial flavors, gluten, or gelatin. In certain embodiments, the gummy form can be substantially free of high fructose corn syrup, artificial sweeteners, artificial flavors, gluten, and gelatin.

In certain embodiments, the gummy form can further include zinc, melatonin, Vitamin C, elderberry, thyme, or combinations thereof. In certain embodiments, the gummy form can further include a sweetener selected from the group consisting of glycose syrup, stevia Rebaudioside A, Stevia leaf extract, monk fruit, sucrose, fructose, tapioca syrup, dextrose, and combinations thereof.

In certain embodiments, the gummy form can be substantially free of an artificial sweetener.

In certain embodiments the gummy form can have a mass of from about 1 gram to about 8 grams. In certain embodiments, the gummy form can have a mass of from about 4 grams to about 6 grams. In certain embodiments, the gummy form can have a mass of about 5 grams.

The present disclosure further provides a method of treatment of a subject suffering from a cough or upper respiratory condition by use of the gummy form of the present disclosure.

In certain embodiments, the subject can be suffering from a cough. The cough can be due to hoarseness, dry throat, irritants, or a combination thereof.

In certain embodiments, the subject can be suffering from difficulty sleeping from the cough, and the gummy form can include melatonin.

### 4. DETAILED DESCRIPTION

The presently disclosed subject matter relates to improved gummy dosage forms including marshmallow root, an ingredient indicated as a natural and herbal ingredient for the treatment of upper respiratory conditions such as cough. Gummy dosage forms inherently have a high water content, however, much of the water can be bound in the gelling matrix through hydration of the gelling materials. The center portion of a gummy dosage form in a center-filled gummy also can have a high level of water. A high level of water in the liquid center fill can interact and hydrate marshmallow root, adversely affecting the texture and consumer experience of the gummy dosage form when administered. This requires at least partial separation of the marshmallow root in a different portion of the center-filled gummy dosage form.

Gummy dosage forms of the present disclosure and methods of making the same advantageously ensure the texture of a liquid or semi-solid center fill portion while retaining a firmer outer gummy shell portion and therefore provide a convenient and pleasant dosage form to consumers, for example, for the treatment of upper respiratory conditions such as cough. Incorporation of a majority portion of the therapeutic ingredient marshmallow root into the outer gummy shell portion was surprisingly and advantageously found to reduce or minimize hydration of the marshmallow root, such that the center portion can be retained as a liquid or semi-solid. Additionally, gummy dosage forms of the present disclosure can include one or more additional ingredients, for example, to treat upper respiratory conditions such as cough (e.g., honey). Such ingredients can be incorporated, for example, into the liquid or semi-solid center fill portion of the gummy dosage form.

Methods of using gummy dosage forms as disclosed herein are further provided and include the treatment of a subject suffering from cough or an upper respiratory condition, such as cold, congestion, allergy, or flu. In certain embodiments, the cough can be due to hoarseness, dry throat, irritants, or a combination thereof. In certain embodiments, the subject can be suffering from difficulty sleeping from the cough, and the gummy dosage form can include melatonin.

These and other aspects of the disclosed subject matter are discussed in more detail below and in the Examples. For clarity and not by way of limitation, this detailed description is divided into the following sub-portions:
4.1. Definitions;
4.2. Gummy Dosage Forms;
4.3. Methods of Making Gummy Dosage Forms;
4.4. Methods of Using Gummy Dosage Forms;
4.5. Features of Gummy Dosage Forms; and
4.6. Other Dosage Forms.

### 4.1. Definitions

The terms used in this specification generally have their ordinary meanings in the art within the context of this disclosure and in specific context where each term is used. Certain terms are discussed below, or elsewhere in the specification, to provide additional guidance in describing the compositions and methods of the disclosure and how to make and use them.

As used in the specification and the appended claims, the singular forms “a,” “an,” and “the” include the plural referents unless the context clearly dictates otherwise. Thus, for example, reference to “an ingredient” includes mixtures of ingredients.

As used herein, the terms “about” or “approximately” mean within an acceptable error range for the particular value as determined by one of ordinary skill in the art, which will depend in part on how the value is measured or determined (i.e., the limitations of the measurement system). For example, “about” can mean within three or more standard deviations per practice in the art. Alternatively, “about” can mean a range of up to 10%, up to 5%, up to 3%, up to 1%, or up to 0.5% of a given value. Particularly with respect to systems or processes, the term can mean within an order of magnitude, for example, within five-fold or within two-fold of a value.

As used herein, the term “center” refers to an inner portion of the gummy dosage form.

As used herein, the terms “comprises,” “comprising,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

As used herein, the terms “dosage,” “dosage form,” “dose unit,” or “dose” refers to the amount of a therapeutic ingredient comprising therapeutically active agent(s) administered at a time. The terms “dosage,” “dosage form,” “dose unit,” or “dose” includes administration of one or more units of therapeutic ingredient administered at the same time. A therapeutic ingredient can include, for example, a pharmaceutical, natural, supplement or herbal ingredient.

As used herein, the term “gelling matrix” refers to the portion of the gummy shell of the gummy dosage form wherein the gelling agents are hydrated, deposited and formed, including the bound water.

As used herein, the terms “gummy form” or “gummy dosage form” refers to a dosage form which retains its integrity and texture upon chewing, does not break into discrete, solid particulates upon chewing, utilizes a gelling matrix, and is intended to be swallowed. The gummy form or gummy dosage form can be palatable, edible, and can be similar in texture to confectionery jelly beans.

As used herein, the term “shell” refers to an outer portion of the gummy dosage form.

As used herein, the terms “% w/w” or “weight percent” refers to the percentage of an ingredient(s)/the total percentage by weight of the composition (100%). The terms “% w/w” or “weight percent” refer to the quantity by weight of a constituent or component, for example, in a gummy dosage form as a percentage of the overall weight of the gummy dosage form. The terms “% w/w” or “weight percent” can also refer to the quantity by weight of a constituent or component, for example in an outer gummy shell portion of the gummy dosage form as a percentage by weight of the outer gummy shell portion. The terms “weight percent”, “wt-%”, “wt. %”, and “wt %” are used interchangeably.

### 4.2. Gummy Dosage Forms

Gummy dosage forms of the present disclosure advantageously provide a liquid or semi-solid center portion while retaining a firmer outer portion in a convenient and pleasant dosage form for delivery of a therapeutic ingredient such as marshmallow root. Such gummy dosage forms can have multiple components including at least a center portion and an outer portion. In certain embodiments, at least one portion of the gummy dosage form can include one or more therapeutic ingredients. In certain embodiments, the one or more therapeutic ingredients can be taste masked. Taste masking technologies are well known for a person skilled in the art. The center portion can be a liquid or semi-solid and can include honey. The outer portion can be a semi-solid or solid and can include marshmallow root. In certain aspects, the gummy dosage forms of the present disclosure can have one or more textures or a dual texture. For example, a texture of the center portion can be different than a texture of the outer portion. The outer portion can at least partially cover the center portion. In certain embodiments, the outer portion can wholly cover or encompass the center portion. In certain embodiments, the gummy dosage form can further include a coating.

### 4.2.1. Center Portion

In certain embodiments, gummy dosage forms of the present disclosure can be a multi-component form including a center portion. Such center portion can be a liquid (e.g., solution, suspension, emulsion, oil, or cream) or a semi-solid. In certain embodiments, the center portion can include a liquid. In certain embodiments, the center portion can include a semi-solid. In certain embodiments, the center portion can include a solution, suspension, emulsion, or semi-solid. The center portion can be present in various states in which the texture is different from the outer portion of the gummy dosage form. In certain embodiments, a texture of the center portion can be different than a texture of the outer portion. In particular embodiments, a texture of the center portion can be less firm than a texture of the outer portion.

In certain embodiments, the center portion can include honey, one or more flavorings, or a combination thereof.

### 4.2.1.1. Honey

In certain embodiments, the center portion of the gummy dosage form can include honey. Any suitable type of honey can be used in the center portion of gummy dosage forms of the present disclosure. In certain embodimens, the center portion of the gummy dosage form can include U.S. Grade A honey (e.g., with a color level of Light Amber to Dark Amber), Buckwheat honey, Wildflower honey, and combinations thereof.

In certain embodiments, the center portion can include honey in an amount of from about 0.2 grams to about 3 grams, about 0.5 grams to about 2 grams, or about 1 gram to about 3 grams. In particular embodiments, the center portion can include about 0.2 grams, about 0.5 grams, about 0.8 grams, about 1 gram, about 1.5 grams, about 2 grams, about 2.5 grams, or about 3 grams of honey. In certain embodiments, the center portion can include honey in an amount of from about 1 mL to about 2.5 mL, about 1 mL to about 3 mL, about 1 mL to about 3.5 mL, about 1 mL to about 4 mL, about 1.5 mL to about 2.5 mL, about 1.5 mL to about 3 mL, about 1.5 mL to about 3.5 mL, about 1.5 mL to about 4 mL, about 2 mL to about 2.5 mL, about 2 mL to about 3 mL, about 2 mL to about 3.5 mL, about 2 mL to about 4 mL, about 2.5 mL to about 3 mL, about 2.5 mL to about 3.5 mL, or about 2.5 mL to about 4 mL. In certain embodiments, the center portion can include about 1 mL, about 1.5 mL, about 2 mL, about 2.5 mL, about 3 mL, about 3.5 mL, or about 4 mL of honey.

In certain embodiments, the center portion can include honey in an amount of from about 50% to about 100%, about 50% to about 99.9%, about 50% to about 99%, about 60% to about 99%, about 75% to about 95%, or about 90% to about 100% by weight of honey, based on the total weight of the center portion. In certain embodiments, the center portion can include at least about 50%, at least about 60%, at least about 75%, at least about 80%, at least about 90%, at least about 95%, or at least about 99% by weight honey, based on the total weight of the center portion. In certain embodiments, the center portion can include about 50%, about 60%, about 75%, about 80%, about 90%, about 95%, about 99%, about 99.95%, or about 100% by weight honey, based on the total weight of the center portion.

### 4.2.1.2. Flavorings

In certain embodiments, the center portion can include one or more flavorings. A person skilled in the art will appreciate a wide variety of flavorings are suitable for use with the present disclosure. The one or more flavorings can include any suitable flavorings, for example, and not by way of limitation, peppermint, spearmint, eucalyptus, licorice, vanilla, caramel, mixed berries, mixed fruits, black current, blueberry, cherry, lemon, or combinations thereof.

In certain embodiments, the center portion can include one or more flavorings in an amount of from about 0.0001 grams to about 2 grams, about 0.0001 grams to about 1 gram, about 0.0001 grams to about 0.5 grams, or about 0.0001 grams to about 0.0005 grams. In certain embodiments, the center portion can include one or more flavorings in an amount of about 0.0001 grams, about 0.0005 grams, about 0.001 grams, about 0.005 grams, about 0.01 grams, about 0.05 grams, about 0.1 grams, about 0.5 grams, about 1 gram, about 1.5 grams, or about 2 grams. In certain embodiments, the center portion can include one or more flavorings in an amount of from about 0.01% to about 2%, about 0.01% to about 1.5%, about 0.01% to about 1%, or about 0.05% to about 1% by weight, based on the total weight of the center portion. In certain embodiments, the center portion can include one or more flavorings in an amount of about 0.01%, about 0.05%, about 0.08%, about 0.1%, about 0.5%, about 1%, about 1.5%, or about 2% by weight, based on the total weight of the center portion.

### 4.2.1.3. Marshmallow Root

In certain embodiments, the center portion of the gummy dosage form can be substantially free of marshmallow root. As used herein, “substantially free” is defined as less than about 0.001% by weight of the center portion of the gummy dosage form.

### 4.2.1.4. Center Portion Formulations

In certain embodiments, the center portion of the gummy dosage form can include honey and one or more flavorings. The honey can be present in amount of from about 50% to about 99% by weight, based on the total weight of the center portion. The one or more flavorings can be present in an amount of from about 0.01% to about 2% by weight, based on the total weight of the center portion. In particular embodiments, the center portion can include about 99.95% by weight honey and about 0.05% by weight of one or more flavorings, based on the total weight of the center portion. In certain embodiments, the center portion can be substantially free of marshmallow root.

### 4.2.2. Outer Portion

In certain embodiments, gummy dosage forms of the present disclosure can be a multi-component form including an outer portion. The outer portion can be semi-solid or solid. Such outer portion can at least partially cover the center portion. In certain embodiments, the outer portion can wholly encompass or cover the center portion. The outer portion can include marshmallow root, one or more gelling agents, one or more acidulants, one or more sweeteners, one or more additional natural or herbal ingredients, water, or combinations thereof.

### 4.2.2.1. Marshmallow Root

In certain embodiments, the outer portion of the gummy dosage form can include marshmallow root. The marshmallow root can be incorporated in the form of a liquid extract or a powder. In certain embodiments, marshmallow root can be present in the outer portion in an amount of from about 0.01 grams to about 10 grams, about 0.5 grams to about 8 grams, or about 0.05 grams to about 5 grams. In certain embodiments, the outer portion can include marshmallow root in an amount of about 0.01 grams, about 0.025 grams, about 0.5 grams, about 1 gram, about 1.5 grams, about 2 grams, about 5 grams, about 8 grams, or about 10 grams. In certain embodiments, the marshmallow root can be present in an amount of from about 0.01% to about 10%, about 0.5% to about 5%, or about 0.5% to about 1% by weight, based on the total weight of the outer portion. In certain embodiments, the marshmallow root can be present in an amount of about 0.01%, about 0.5%, about 0.6%, about 0.8%, about 1%, about 1.5%, about 2%, about 5%, about 8%, or about 10% by weight, based on the total weight of the outer portion. In certain embodiments, the outer portion can include at least about 0.1%, at least about 0.2%, at least about 0.4%, at least about 0.5%, at least about 0.6%, at least about 0.8%, at least about 1%, or at least about 5% by weight of marshmallow root, based on the total weight of the outer portion.

In certain embodiments, a majority portion of the marshmallow root can be included into the outer portion. As used herein, the term “majority portion” refers to greater than 50% of the total amount of marshmallow root is present in the outer portion, or at least 51%. In certain embodiments, the majority portion can be greater than 60% or greater than 70% of the total amount of marshmallow root is present in the outer portion.

### 4.2.2.2. Gelling Agents

In certain embodiments, the outer portion of the gummy dosage form can include one or more gelling agents. A person skilled in the art will appreciate a wide variety of gelling agents are suitable for use with the present disclosure. Suitable gelling agents can include, but are not limited to, pectin, starches, celluloses, derived starches, gelatin, carrageenan, alginate, gums, and combinations thereof. In certain embodiments, the one or more gelling agents can include gelatin, pectin, kappa carrageenan, iota carrageenan, a combination of kappa and iota carragenan, or combinations thereof. In certain embodiments, the one or more gelling agents can include pectin. A person skilled in the art will appreciate a wide variety of gums are suitable for use with the present disclosure. Suitable gums include gellan gum, xanthan gum, guar gum, locust bean gum, tragacanth, acacia gum, and combinations thereof. In certain embodiments, more than one gelling agent can be used in the outer portion. In certain embodiments, the gelling matrix of the outer portion can be created when the one or more gelling agents are dissolved into hot water, other ingredients are added, and the form is set in a mold and allowed to cool.

The outer portion can include the one or more gelling agents in an amount of from about 0.05% to about 10%, about 1% to about 8%, about 3% to about 5%, or about 3.5% to about 4% by weight, based on the total weight of the outer portion. In certain embodiments, the outer portion can include the one or more gelling agents in an amount of about 0.05%, about 0.1%, about 0.5%, about 1%, about 3%, about 3.5%, about 4%, about 5%, about 8%, or about 10% by weight, based on the total weight of the outer portion.

### 4.2.2.3. Acidulants

In certain embodiments, the outer portion of the gummy dosage form can include one or more acidulants. A person skilled in the art will appreciate a wide variety of acidulants are suitable for use with the present disclosure. Suitable acidulants include, for example, and not by way of limitation, organic acids such as citric acid, malic acid, fumaric acid, or combinations thereof. In certain embodiments, the one or more acidulants can include citric acid, sodium citrate, or combinations thereof.

In certain embodiments, the outer portion can include the one or more acidulants present in an amount of from about 0.1% to about 10%, about 0.1% to about 5%, about 0.5% to about 3%, about 0.5% to about 2%, or about 1% to about 1.5% by weight, based on the total weight of the outer portion. In certain embodiments, the outer portion can include the one or more acidulants in an amount of about 0.1%, about 0.5%, about 1%, about 1.2%, about 1.5%, about 2%, about 2.5%, about 3%, about 4%, about 5%, about 8%, or about 10% by weight, based on the total weight of the outer portion.

### 4.2.2.4. Sweeteners

In certain embodiments, the outer portion of the gummy dosage form can include one or more sweeteners. A person skilled in the art will appreciate a wide variety of sweeteners are suitable for use with the present disclosure. Suitable sweeteners can include, for example, and not by way of limitation, synthetic or natural sugars; sugars extracted from sugar cane and sugar beet (sucrose), dextrose (also called glucose), fructose (also called laevulose), and lactose (also called milk sugar); isomalt; corn syrup, high fructose corn syrup, stevia, and mixtures thereof. In certain embodiments, the gummy dosage form can include one or more sweeteners selected from the group consisting of glycose syrup, stevia Rebaudioside A, Stevia leaf extract, monk fruit, sucrose, fructose, tapioca syrup, dextrose, and combinations thereof. In certain embodiments, the one or more sweeteners can include sucrose, glucose syrup, or combinations thereof.

In certain embodiments, the outer portion can include the one or more sweeteners in an amount of from about 0.02% to about 90%, about 0.5% to about 90%, about 5% to about 75%, about 10% to about 50%, about 50% to about 90%, about 40% to about 80%, or about 50% to about 75% by weight, based on the total weight of the outer portion. In particular embodiments, the outer portion can include the one or more sweeteners in an amount of about 0.02%, about 0.5%, about 5%, about 10%, about 15%, about 20%, about 25%, about 30%, about 35%, about 40%, about 45%, about 50%, about 55%, about 60%, about 65%, about 70%, about 74% about 75%, about 80%, about 85%, or about 90% by weight, based on the total weight of outer portion. In certain embodiments, the outer portion can include the one or more sweeteners in an amount of at least about 15%, at least about 25%, at least about 40%, at least about 50%, at least about 60%, or at least about 70% by weight, based on the total weight of the outer portion.

In certain embodiments, the one or more sweeteners included in the outer portion can be a high intensity sweetener, for example, stevia, sucralose, or neotame. In such embodiments, the outer portion can include the one or more sweeteners in an amount of from about 0.02% to about 3%, about 0.1% to about 1%, about 0.5% to about 3%, about 0.5% to about 2.5%, or about 1% to about 2% by weight, based on the total weight of the outer portion. In particular embodiments, the outer portion can include the one or more sweeteners in an amount of about 0.02%, about 0.05%, about 0.1%, about 0.2%, about 0.5%, about 0.8%, about 1%, about 1.5%, about 2%, about 2.5%, or about 3% by weight, based on the total weight of the outer portion.

In certain embodiments the one or more sweeteners included in the outer portion can be a bulk sweetener, for example, dextrose. In such embodiments, the outer portion can include the one or more sweeteners in an amount of from about 10% to about 90%, about 15% to about 75%, about 25% to about 50%, about 50% to about 90%, or about 75% to about 90% by weight, based on the total weight of the outer portion. In particular embodiments, the outer portion can include the one or more sweeteners in an amount of about 10%, about 15%, about 20%, about 25%, about 30%, about 40%, about 45%, about 50%, about 55%, about 60%, about 65%, about 70%, about 75%, about 80%, about 85%, or about 90% by weight, based on the total weight of the outer portion.

In certain embodiments, the outer portion can include more than one sweetener. In such embodiments, the outer portion can include at least a first sweetener in an amount of from about 10% to about 50%, about 20% to about 40%, or about 25% to about 35% by weight, based on the total weight of the outer portion. In certain embodiments, the outer portion can further include a second sweetener in an amount of from about 25% to about 65%, about 30% to about 50%, or about 35% to about 45% by weight, based on the total weight of the outer portion.

### 4.2.2.5. Additional Natural or Herbal Ingredients

In certain embodiments, the outer portion of the gummy dosage form can include one or more additional natural or herbal ingredients. A person skilled in the art will appreciate a wide variety of additional natural or herbal ingredients are suitable for use with the present disclosure. In certain embodiments, such ingredients can be complimentary for cough or cold purposes, for example, and not by way of limitation, including Vitamin C (ascorbic acid), echinacea, zinc, turmeric, citrus extracts, African geranium root, eucalyptus, Vitamin D, elderberry (e.g., as elderberry extract), or combinations thereof. In certain embodiments, such ingredients can be complimentary for the purposes of sleep, for example, and not by way of limitation, including melatonin, chamomile, lemon balm, valerian root, or combinations thereof. In certain embodiments, the one or more additional natural or herbal ingredients can include zinc, melatonin, Vitamin C (ascorbic acid), elderberry, thyme, or combinations thereof. In certain embodiments, the one or more additional natural or herbal ingredients can include Vitamin C (ascorbic acid), elderberry (e.g., as elderberry extract), thyme, and combinations thereof.

In certain embodiments, the outer portion can include the one or more additional natural or herbal ingredients in an amount of from about 0.1% to about 40%, about 1% to about 30%, about 5% to about 25%, or about 10% to about 20% by weight, based on the total weight of the outer portion. In certain embodiments, the outer portion can include the one or more additional natural or herbal ingredients in an amount of about 0.1%, about 0.5%, about 1%, about 5%, about 10%, about 11%, about 15%, about 20%, about 25%, about 30%, about 35%, or about 40% by weight, based on the total weight of the outer portion.

### 4.2.2.6. Honey

In certain embodiments, the outer portion of the gummy dosage form can include honey. Any suitable type of honey can be used in the outer portion of gummy dosage forms of the present disclosure. In certain embodimens, the center portion of the gummy dosage form can include U.S. Grade A honey (e.g., with a color level of Light Amber to Dark Amber), Buckwheat honey, Wildflower honey, and combinations thereof.

In certain embodiments, the outer portion can include honey in an amount of from about 0.2 grams to about 3 grams, or from about 1 gram to about 3 grams. In particular embodiments, the outer portion can include about 0.2 grams, about 0.5 grams, about 0.8 grams, about 1 gram, about 1.5 grams, about 2 grams, about 2.5 grams, or about 3 grams of honey. In certain embodiments, the outer portion can include honey in an amount of from about 30% to about 70%, about 40% to about 65%, or from about 50% to about 60% by weight, based on the total weight of the outer portion. In certain embodiments, the outer portion can include honey in an amount of about 70% or less, about 65% or less, about 55% or less, about 50% or less, about 40% or less, or about 30% or less by weight, based on the total weight of the outer portion. In particular embodiments, the outer portion can include honey in an amount of about 30%, about 40%, about 50%, about 55%, about 60%, or about 70% by weight, based on the total weight of the outer portion.

### 4.2.2.7. Water

In certain embodiments, the outer portion can include water. In certain embodiments, the outer portion can include from about 1% to about 20%, about 5% to about 15%, or about 5% to about 10% by weight water, based on the total weight of the outer portion. In certain embodiments, the outer portion can include about 1%, about 5%, about 8%, about 9%, or about 10% by weight water, based on the total weight of the outer portion.

### 4.2.2.8. Outer Portion Formulations

In certain embodiments, the outer portion can include marshmallow root, one or more gelling agents, one or more acidulants, one or more sweeteners, one or more additional natural or herbal ingredients, water, or combinations thereof.

In certain embodiments, the outer portion can include marshmallow root in an amount of from about 0.01% to about 10% by weight; one or more gelling agents in an amount of from about 0.05% to about 10% by weight; one or more acidulants in an amount of from about 0.1% to about 10% by weight; one or more sweeteners in amount of from about 0.02% to about 90% by weight; one or more additional natural or herbal ingredients in an amount of from about 0.1% to about 40% by weight; and water in an amount of from about 1% to about 20% by weight, each based on the total weight of the outer portion. The one or more gelling agents can include pectin. The one or more acidulants can include citric acid (e.g., in a 50% solution with purified water), sodium citrate, or combinations thereof. The one or more natural or herbal ingredients can include Vitamin C (ascorbic acid), elderberry (e.g., as elderberry extract), thyme, or combinations thereof.

### 4.2.3. Coating

In certain embodiments, the gummy dosage form of the present disclosure can include a coating. The coating can be disposed on at least a portion of the outer portion of the gummy dosage form. In certain embodiments, the coating can wholly encompass the outer portion. The coating can be disposed on gummy dosage forms of the present disclosure by various suitable means as known by those skilled in the art. In certain embodiments, the coating can be homogeneous. The coating can be present and applied in an amount of from about 0.5% to about 10%, about 0.5% to about 8%, about 1% to about 5%, or about 1% to about 3% by weight, based on the total weight of the gummy dosage form. In particular embodiments, the coating can be present and applied in an amount of about 0.5%, about 1%, about 2%, about 3%, about 4%, about 5%, about 8%, or about 10% by weight, based on the total weight of the gummy dosage form.

The coating can include various ingredients as known in the art. In certain embodiments, the coating can include one or more sugars, or one or more sugar alcohols. For example, and not by way of limitation, materials suitable for sugar or sugar alcohol-based coatings can include sucrose, dextrose, xylitol, or combinations thereof. In certain embodiments, the coating can be a film coating including one or more polymers. For example, and not by way of limitation, polymers suitable for film coatings can include hypromellose and polyvinyl alcohol, polyvinyl alcohol:polyethylene glycol co-polymers, and mixtures thereof.

### 4.2.4. Additional Additives

Gummy dosage forms of the presently disclosed subject matter can include additional additives in the center portion and/or the outer portion thereof. For example, the gummy dosage form can include, in the center and/or outer portions, one or more of additional natural or herbal ingredients, gelling agents, fats, proteins, colorings, flavorings, sweeteners, acidulants, thickeners, emulsifiers, antioxidants, preservatives, lubricants, glidants, disintegrants, or combinations thereof.

### 4.2.4.1. Additional Natural or Herbal Ingredients

In certain embodiments, the gummy dosage form can include additional therapeutic ingredients to marshmallow root, such as one or more additional natural or herbal ingredients. A person skilled in the art will appreciate a wide variety of natural or herbal ingredients are suitable for use with the present disclosure. In certain embodiments, such ingredients can be complimentary for cough or cold purposes, for example, and not by way of limitation, including Vitamin C (ascorbic acid), echinacea, zinc, turmeric, citrus extracts, African geranium root, eucalyptus, Vitamin D, elderberry (e.g., as elderberry extract), or combinations thereof. In certain embodiments, such ingredients can be complimentary for the purposes of sleep, for example, and not by way of limitation, including melatonin, chamomile, lemon balm, valerian root, or combinations thereof. In certain embodiments, the gummy dosage form can include zinc, melatonin, Vitamin C (ascorbic acid), elderberry, thyme, or combinations thereof.

In certain embodiments, the gummy dosage form can include the one or more additional natural or herbal ingredients in an amount of from about 0.1% to about 40%, about 0.5% to about 35%, about 1% to about 30%, about 5% to about 40%, or about 10% to about 30% by weight, based on the total weight of the gummy dosage form. In particular embodiments, the gummy dosage form can include the one or more additional natural or herbal ingredients in an amount of about 0.1%, about 0.5%, about 1%, about 5%, about 10%, about 15%, about 20%, about 25%, about 30%, about 35%, or about 40% by weight, based on the total weight of the gummy dosage form. A person skilled in the art will appreciate an amount of the one or more additional natural or herbal ingredients can widely vary, for example, depending on the actual dose.

### 4.2.4.2. Flavorings

In certain embodiments, the gummy dosage form can include one or more flavorings. A person skilled in the art will appreciate a wide variety of flavorings are suitable for use with the present disclosure. The one or more flavorings can include any suitable flavorings, for example, and not by way of limitation, peppermint, spearmint, eucalyptus, licorice, vanilla, caramel, mixed berries, mixed fruits, black current, blueberry, cherry, lemon, or combinations thereof.

In certain embodiments, the gummy dosage form can include the one or more flavorings in an amount of from about 0.01% to about 5%, about 0.01% to about 3%, about 0.1% to about 3%, about 0.5% to about 1.5%, or about 1% to about 3% by weight, based on the total weight of the gummy dosage form. In particular embodiments, the gummy dosage form can include the one or more flavorings in an amount of about 0.01%, about 0.05%, about 0.08%, about 0.1%, about 0.5%, about 0.8%, about 1%, about 1.5%, about 2%, about 2.5%, about 3%, about 3.5%, about 4%, about 4.5%, or about 5% by weight, based on the total weight of the gummy dosage form.

### 4.2.4.3. Acidulants

In certain embodiments, the gummy dosage form can include one or more acidulants. A person skilled in the art will appreciate a wide variety of acidulants are suitable for use with the present disclosure. Suitable acidulants can include, for example, and not by way of limitation, organic acids such as citric acid, malic acid, fumaric acid, or combinations thereof. In certain embodiments, the one or more acidulants can include citric acid, sodium citrate, or combinations thereof.

In certain embodiments, the gummy dosage form can include the one or more acidulants present in an amount of from about 0.1% to about 10%, about 0.1% to about 5%, about 0.5% to about 3%, about 0.5% to about 2%, or about 1% to about 1.5% by weight, based on the total weight of the gummy dosage form. In certain embodiments, the gummy dosage form can include the one or more acidulants in an amount of about 0.1%, about 0.5%, about 1%, about 1.2%, about 1.5%, about 2%, about 2.5%, about 3%, about 4%, about 5%, about 8%, or about 10% by weight, based on the total weight of the gummy dosage form.

### 4.2.4.4. Solvents

In certain embodiments, the gummy dosage form can include one or more solvents. A person skilled in the art will appreciate a wide variety of solvents are suitable for use with the present disclosure. Suitable solvents can include, for example, and not by way of limitation, propylene glycol, polyethylene glycol, glycerin, or combinations thereof. In particular embodiments, the outer portion of the gummy dosage form can include one or more solvents.

In certain embodiments, the gummy dosage form can include the one or more solvents in an amount of from about 2% to about 30%, about 5% to about 25%, about 10% to about 30% or about 5% to about 15% by weight, based on the total weight of the gummy dosage form.

### 4.2.4.5. Excipients

In certain embodiments, the gummy dosage form can include one or more excipients. A person skilled in the art will appreciate a wide variety of excipients are suitable for use with the present disclosure. Suitable excipients can include, for example, and not by way of limitation, fats, proteins, fillers, glidants, lubricants, sweeteners, flavors, coloring agents, fillers, binding/gelling agents, and mixtures thereof.

In certain embodiments, the gummy dosage form can include the one or more excipients in an amount of from about 50% to about 99%, about 60% to about 95%, about 70% to about 99%, about 80% to about 99%, or about 85% to about 95% by weight, based on the total weight of the gummy dosage form.

### Lubricants

In certain embodiments, the gummy dosage form can include one or more lubricants. A person skilled in the art will appreciate a wide variety of lubricants are suitable for use with the present disclosure. Suitable lubricants can include, for example, and not by way of limitation, long chain fatty acids and their salts, such as magnesium stearate and stearic acid, talc, glycerides waxes, and mixtures thereof.

### Glidants

In certain embodiments, the gummy dosage form can include one or more glidants. A person skilled in the art will appreciate a wide variety of glidants are suitable for use with the present disclosure. Suitable glidants can include, for example, and not by way of limitation, colloidal silicon dioxide.

In certain embodiments, the gummy dosage form can include the one or more glidants in an amount of from about 0.1% to about 3%, about 0.5% to about 2.5%, about 1% to about 3%, or about 1.5% to about 2.5% by weight, based on the total weight of the gummy dosage form. In particular embodiments, the gummy dosage form can include the one or more glidants in an amount of about 0.1%, about 0.5%, about 1%, about 1.5%, about 2%, about 2.5%, or about 3% by weight, based on the total weight of the gummy dosage form.

### Gelling Agents

In certain embodiments, the gummy dosage form can include one or more gelling agents. A person skilled in the art will appreciate a wide variety of gelling agents are suitable for use with the present disclosure. Suitable gelling agents can include, but are not limited to, pectin, starches, celluloses, derived starches, gelatin, carrageenan, alginate, gums, and combinations thereof. In certain embodiments, the one or more gelling agents can include gelatin, pectin, kappa carrageenan, iota carrageenan, a combination of kappa and iota carragenan, or combinations thereof. In certain embodiments, the one or more gelling agents can include pectin. A person skilled in the art will appreciate a wide variety of gums are suitable for use with the present disclosure. Suitable gums can include gellan gum, xanthan gum, guar gum, locust bean gum, tragacanth, acacia gum, and combinations thereof.

In certain embodiments, the gummy dosage form can include the one or more gelling agents in an amount of from about 0.5% to about 30%, about 1% to about 25%, or about 5% to about 15% by weight, based on the total weight of the gummy dosage form. In particular embodiments, the gummy dosage form can include the one or more glidants in an amount of about 0.5%, about 1%, about 5%, about 10%, about 15%, about 20%, about 25%, or about 30% by weight, based on the total weight of the gummy dosage form.

### Sweeteners

In certain embodiments, the gummy dosage form can include one or more sweeteners. A person skilled in the art will appreciate a wide variety of sweeteners are suitable for use with the present disclosure. Suitable sweeteners can include, for example, and not by way of limitation, synthetic or natural sugars; sugars extracted from sugar cane and sugar beet (sucrose), dextrose (also called glucose), fructose (also called laevulose), and lactose (also called milk sugar); isomalt; corn syrup, high fructose corn syrup, stevia, and mixtures thereof.

In certain embodiments, the gummy dosage form can be substantially free of artificial sweeteners. In certain embodiments, the gummy dosage form can include one or more sweeteners selected from the group consisting of glycose syrup, stevia Rebaudioside A, Stevia leaf extract, monk fruit, sucrose, fructose, tapioca syrup, dextrose, and combinations thereof. In certain embodiments, the one or more sweeteners can include sucrose, glucose syrup, or combinations thereof.

In certain embodiments, the gummy dosage form can include the one or more sweeteners present in an amount of from about 0.5% to about 90%, about 5% to about 75%, about 10% to about 50%, or about 50% to about 90% by weight, based on the total weight of the gummy dosage form. In particular embodiments, the gummy dosage form can include the one or more sweeteners in an amount of about 0.5%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% by weight, based on the total weight of the gummy dosage form. In certain embodiments, the gummy dosage form can include the one or more sweeteners in an amount of at least about 15%, at least about 25%, at least about 40%, at least about 50%, at least about 60%, or at least about 70% by weight, based on the total weight of the gummy dosage form.

In certain embodiments, the one or more sweeteners included in the gummy dosage form can be a high intensity sweetener, for example, stevia, sucralose, or neotame. In such embodiments, the gummy dosage form can include the one or more sweeteners in an amount of from about 0.02% to about 3%, about 0.1% to about 1%, about 0.5% to about 3%, about 0.5% to about 2.5%, or about 1% to about 2% by weight, based on the total weight of the gummy dosage form. In particular embodiments, the gummy dosage form can include the one or more sweeteners in an amount of about 0.02%, about 0.05%, about 0.1%, about 0.2%, about 0.5%, about 0.8%, about 1%, about 1.5%, about 2%, about 2.5%, or about 3% by weight, based on the total weight of the gummy dosage form.

In certain embodiments the one or more sweeteners included in the gummy dosage form can be a bulk sweetener, for example, dextrose. In such embodiments, the gummy dosage form can include the one or more sweeteners in an amount of from about 10% to about 90%, about 15% to about 75%, about 25% to about 50%, about 50% to about 90%, or about 75% to about 90% by weight, based on the total weight of the gummy dosage form. In particular embodiments, the gummy dosage form can include the one or more sweeteners in an amount of about 10%, about 15%, about 20%, about 25%, about 30%, about 40%, about 45%, about 50%, about 55%, about 60%, about 65%, about 70%, about 75%, about 80%, about 85%, or about 90% by weight, based on the total weight of the gummy dosage form.

### Coloring Agents

In certain embodiments, the gummy dosage form can include one or more coloring agents. A person skilled in the art will appreciate a wide variety of coloring agents are suitable for use with the present disclosure. Suitable coloring agents can include, for example, and not by way of limitation, lakes and dyes approved as a food additive, including natural coloring agents, and combinations thereof.

In certain embodiments, the gummy dosage form can include the one or more coloring agents in an amount of from about 0.01% to about 5%, about 0.05% to about 3%, about 0.5% to about 2%, or about 1% to about 3% by weight, based on the total weight of the gummy dosage form. In particular embodiments, the gummy dosage form can include the one or more coloring agents in an amount of about 0.01%, about 0.05%, about 0.1%, about 0.5%, about 1%, about 1.5%, about 2%, about 3%, about 4%, or about 5% by weight, based on the total weight of the gummy dosage form.

### Fillers

In certain embodiments, the gummy dosage form can include one or more fillers. A person skilled in the art will appreciate a wide variety of fillers are suitable for use with the present disclosure. Suitable fillers include corn syrup, sucrose, starches, fats, proteins, gelatin, or combinations thereof. Additional materials that can be used in the gummy dosage form as fillers include, for example, corn syrup solids, sucrose, starches, fats, coconut oil, lecithin, proteins, gelatin, or combinations thereof.

In certain embodiments, the gummy dosage form can include the one or more fillers in an amount of from about 30% to about 95%, about 40% to about 80%, about 35% to about 75%, or about 50% to about 65% by weight, based on the total weight of the gummy dosage form. In particular embodiments, the gummy dosage form can include the one or more fillers in an amount of about 30%, about 35%, about 40%, about 45%, about 50%, about 55%, about 60%, about 65%, about 70%, about 75%, about 80%, about 85%, about 90%, or about 95% by weight, based on the total weight of the gummy dosage form.

### Fats

In certain embodiments, the gummy dosage form can include one or more fats. A person skilled in the art will appreciate a wide variety of fats are suitable for use with the present disclosure.

In certain embodiments, the gummy dosage form can include one or more fats in an amount of from about 1% to about 30%, about 10% to about 30%, about 5% to about 20%, or about 10% to about 15% by weight, based on the total weight of the gummy dosage form.

### Proteins

In certain embodiments, the gummy dosage form can include one or more proteins. A person skilled in the art will appreciate a wide variety of proteins are suitable for use with the present disclosure.

In certain embodiments, the gummy dosage form can include one or more proteins in an amount of from about 1% to about 20%, about 5% to about 15%, or about 10% to about 20% by weight, based on the total weight of the gummy dosage form. In particular embodiments, the gummy dosage form can include one or more proteins in an amount of about 1%, about 5%, about 8%, about 10%, about 15%, or about 20% by weight, based on the total weight of the gummy dosage form.

### 4.2.5. Gummy Dosage Form Compositions

Gummy dosage forms of the present disclosure can be multi-component compositions including center and outer portions. In certain embodiments, the center portion can include honey, one or more flavorings, or combinations thereof. The outer portion can include marshmallow root. In certain embodiments, the outer portion can further include one or more gelling agents, one or more acidulants, one or more sweeteners, one or more additional natural or herbal ingredients, water, or combinations thereof. In certain embodiments, the center portion can be substantially free of marshmallow root.

The center portion of the gummy dosage form can include honey and one or more flavorings. The honey can be present in amount of from about 50% to about 99% by weight, based on the total weight of the center portion. The one or more flavorings can be present in an amount of from about 0.01% to about 2% by weight, based on the total weight of the center portion. The outer portion can include marshmallow root in an amount of from about 0.01% to about 10% by weight; one or more gelling agents in an amount of from about 0.05% to about 10% by weight; one or more acidulants in an amount of from about 0.1% to about 10% by weight; one or more sweeteners in amount of from about 0.02% to about 90% by weight; one or more additional natural or herbal ingredients in an amount of from about 0.1% to about 40% by weight; and water in an amount of from about 1% to about 20% by weight, each based on the total weight of the outer portion. The one or more gelling agents can include pectin. The one or more acidulants can include citric acid (e.g., in a 50% solution with purified water), sodium citrate, or combinations thereof. The one or more natural or herbal ingredients can include Vitamin C (ascorbic acid), elderberry (e.g., as elderberry extract), thyme, or combinations thereof.

In certain embodiments, the gummy dosage form can be substantially free of one or more of high fructose corn syrup, artificial sweeteners, artificial flavors, gluten, or gelatin. In certain embodiments, the gummy form can be substantially free of high fructose corn syrup, artificial sweeteners, artificial flavors, gluten, and gelatin. In certain embodiments, the gummy dosage form can be substantially free of an artificial sweetener.

### 4.3. Methods of Making Gummy Dosage Forms

In certain embodiments, a method of manufacturing a gummy dosage form of the present disclosure is provided. A wide variety of processes can be used to provide the gummy dosage form of the present disclosure. In certain embodiments, the gummy dosage form can be produced using a starch mold process in which a bed of starch is pre-molded prior to the deposition of a flowable outer shell portion. The outer shell portion is allowed to form and cool. Alternatively, the gummy dosage form can be produced with a non-starch mold, such as a ceramic, silicone, metal, or coated metal mold, in which the outer flowable shell portion is directly deposited in the mold and cooled to form the outer shell portion of the gummy dosage form.

The outer shell portion of the gummy dosage form can be prepared by mixing and heating a solution and depositing the heated solution into a mold. The outer shell portion is allowed to cool to room temperature or approximately 25° C. and is set prior to the depositing of a liquid or semi-solid center portion. The center portion can be kept at a temperature of less than 30° C. so as not to dissolve the outer shell portion when the center portion is incorporated therein. The center portion can be added, for example, through a preformed opening or through injection into the outer shell portion of the gummy dosage form with a canula, needle, or other suitable device fitted with a nozzle and intended to deliver liquids into a confined space.

In certain embodiments, a method of manufacturing a gummy dosage form of the present disclosure is provided. The method can include preparation of the outer portion and the center portion of the gummy dosage form. In certain embodiments, the gelling matrix of the outer portion can be created when the one or more gelling agents are dissolved into hot water, other ingredients are added, and the form is set in a mold and allowed to cool. The outer portion can be prepared, for example, by forming a mixture of a gelling agent, a first acidulant, and a first portion of a first sweetener (e.g., one third), which can be provided as dry ingredients. The dry mixture can be added to water (at 90° C.) to form a solution and stirred until the gelling agent swells. The solution can be brought to a boil to form a boiling mixture. A second portion of the first sweetener (e.g., two thirds) and a second acidulant mixed with purified water to create a 50% solution can both be added to the boiling mixture and mixed until dissolved. In a separate container, a second sweetener can be brought to a boil and added to the other mixture and mixed. Marshmallow root and one or more additional natural or herbal ingredients can be added to the mixture and mixed until dissolved. The resulting mixture can be added to molds and allowed to cool to form the outer portion of the gummy dosage form. In certain embodiments, the molds can allow for a portion of a liquid center portion to be added in a separate step. In certain embodiments, the gelling agent can be pectin, the first acidulant can be sodium citrate, the second acidulant can be citric acid, the first sweetener can be sucrose, and the second sweetener can be glucose syrup. In certain embodiments, the one or more additional natural or herbal ingredients can include Vitamin C (ascorbic acid), elderberry (e.g., elderberry extract), thyme, and combinations thereof. The center portion can be prepared, for example, by forming a mixture of honey and one or more flavorings at room temperature until the one or more flavorings is dissolved. The mixture can be deposited into the outer portion (e.g., by drawing the mixture into a sterile canula and injected into the center portion of the outer portion).

### 4.4. Methods of Using Gummy Dosage Forms

Gummy dosage forms of the present disclosure can be used, for example, alone as a dosage form for delivery of one or more therapeutic ingredients. The gummy dosage forms of the present disclosure can be administered orally. Methods of treatment of a subject suffering from a cough or upper respiratory condition such as cold, congestion, allergy, or flu, by use of the gummy dosage forms described herein are provided. In certain embodiments, the subject can be suffering from a cough which is due to hoarseness, dry throat, irritants, or a combination thereof. In certain embodiments, the subject can be suffering from difficulty sleeping from the cough and the gummy dosage form can include melatonin.

### 4.5. Features of Gummy Dosage Forms

The present disclosure provides for gummy dosage forms. Such gummy dosage forms can be multi-component compositions having center and outer portions in which the outer portion at least partially covers the center portion. The center portion can be a liquid or semi-solid and the outer portion can be semi-solid or solid. The center portion can be present in various states in which the texture is different from the outer portion of the gummy dosage form. The gummy dosage form can therefore have a dual texture.

In certain embodiments, the center portion of the gummy dosage form can be present in an amount of from about 0.5 grams to about 3 grams or from about 10% to about 50% by weight of the gummy dosage form. In certain embodiments, the center portion can be present in an amount of from about 0.5 grams to about 2.5 grams, about 1 gram to about 2 grams, or about 1.5 grams to about 2 grams. In particular embodiments, the center portion can be present in an amount of about 0.5 grams, about 1 gram, about 1.5 grams, about 2 grams, about 2.5 grams, or about 3 grams. In certain embodiments, the center portion can be present in an amount of from about 10% to about 45%, about 15% to about 40%, or about 20% to about 35% by weight of the gummy dosage form. In certain embodiments, the center portion can be present in about of about 10%, about 15%, about 20%, about 25%, about 30%, about 35%, or about 40% by weight of the gummy dosage form.

In certain embodiments, the outer portion of the gummy dosage form can be present in an amount of from about 1 gram to about 6 grams or from about 50% to about 90% by weight of the gummy dosage form. In certain embodiments, the outer portion can be present in an amount of from about 1 gram to about 5.5 grams, about 1.5 grams to about 4.5 grams, or about 2 grams to about 3 grams. In particular embodiments, the outer portion can be present in an amount of about 1 gram, about 1.5 grams, about 2 grams, about 2.5 grams, about 3 grams, about 3.5 grams, about 4 grams, about 4.5 grams, about 5 grams, about 5.5 grams, or about 6 grams. In certain embodiments, the outer portion can be present in an amount of from about 50% to about 85%, about 60% to about 75%, or about 65% to about 75% by weight of the gummy dosage form. In particular embodiments, the outer portion can be present in an about of about 50%, about 55%, about 60%, about 65%, about 70%, about 75%, about 80%, or about 85% by weight of the gummy dosage form.

In certain embodiments, the ratio of the center portion to the outer portion of the gummy dosage form can be about 1:4, about 1:2, or about 1:1.

In certain embodiments, the gummy dosage form can be of a weight which can accommodate a portion of honey and a portion of marshmallow root. The gummy dosage form can have a mass of from about 0.5 grams to about 7 grams or about 2 grams to about 6 grams. In certain embodiments, the gummy dosage form can have a mass of about 1 gram to about 5 grams, about 1 gram to about 5.5 grams, about 1 gram to about 6 grams, about 1 gram to about 6.5 grams, about 1 gram to about 7 grams, about 1 gram to about 7.5 grams, about 1 gram to about 8 grams, about 1.5 grams to about 5 grams, about 1.5 grams to about 5.5 grams, about 1.5 grams to about 6 grams, about 1.5 grams to about 6.5 grams, about 1.5 grams to about 7 grams, about 1.5 grams to about 7.5 grams, about 1.5 grams to about 8 grams, about 2 grams to about 5 grams, about 2 grams to about 5.5 grams, about 2 grams to about 6 grams, about 2 grams to about 6.5 grams, about 2 grams to about 7 grams, about 2 grams to about 7.5 grams, about 2 grams to about 8 grams, about 2.5 grams to about 5 grams, about 2.5 grams to about 5.5 grams, about 2.5 grams to about 6 grams, about 2.5 grams to about 6.5 grams, about 2.5 grams to about 7 grams, about 2.5 grams to about 7.5 grams, about 2.5 grams to about 8 grams, about 3 grams to about 5 grams, about 3 grams to about 5.5 grams, about 3 grams to about 6 grams, about 3 grams to about 6.5 grams, about 3 grams to about 7 grams, about 3 grams to about 7.5 grams, about 3 grams to about 8 grams, about 3.5 grams to about 5 grams, about 3.5 grams to about 5.5 grams, about 3.5 grams to about 6 grams, about 3.5 grams to about 6.5 grams, about 3.5 grams to about 7 grams, about 3.5 grams to about 7.5 grams, about 3.5 grams to about 8 grams, about 4 grams to about 5 grams, about 4 grams to about 5.5 grams, about 4 grams to about 6 grams, about 4 grams to about 6.5 grams, about 4 grams to about 7 grams, about 4 grams to about 7.5 grams, about 4 grams to about 8 grams, about 4.5 grams to about 5 grams, about 4.5 grams to about 5.5 grams, about 4.5 grams to about 6 grams, about 4.5 grams to about 6.5 grams, about 4.5 grams to about 7 grams, about 4.5 grams to about 7.5 grams, about 4.5 grams to about 8 grams, about 5 grams to about 5.5 grams, about 5 grams to about 6 grams, about 5 grams to about 6.5 grams, about 5 grams to about 7 grams, about 5 grams to about 7.5 grams, or about 5 grams to about 8 grams. In particular embodiments, the gummy dosage form can have a mass of about 4.5 grams, about 5 grams, about 5.5 grams, or about 6 grams.

### 4.6. Other Dosage Forms

Compositions of the present disclosure can be utilized in other dosage forms having an outer shell portion and a liquid or semi-solid center portion. Such forms include, for example, lozenges, chewing gums, and soft chewables (“soft chews”). In each of these forms, a majority portion of the marshmallow root can be incorporated into the outer shell portion and not included (or substantially free of) in the liquid or semi-solid center portion. As used herein, the term “majority portion” refers to greater than 50% of the total amount of marshmallow root is present in the outer shell portion, or at least 51%. In certain embodiments, the majority portion can be greater than 60% or greater than 70% of the total amount of marshmallow root is present in the outer shell portion.

Lozenges can have a candy glass or compressed outer shell portion and are intended to dissolve in the oral cavity such as the mouth or pharynx without chewing. For lozenges, the dissolved materials from the dosage form are eventually ingested.

Soft chew forms are intended as dosage forms which retain a portion of their integrity and texture upon chewing, after which they are swallowed. Soft chews can be opaque and contain at least one of corn starch, corn syrup, corn syrup solids, glycerin, coconut oil, palm oil, or lecithin. Soft chews can be differentiated from gummy forms, for example, through the incorporation of a lipid such as palm oil or coconut oil.

Chewing gum forms are intended for chewing in the oral cavity while the therapeutic ingredients contained in the form are released. Chewing gums are not intended to be swallowed. The shell of a chewing gum typically comprises a chewing gum base which further comprises resins and elastomers which are not digestible.

### 5. EXAMPLES

The following Examples are intended to illustrate, but not to limit, the disclosed subject matter in any manner, shape, or form, either explicitly or implicitly.

### Prophetic Example 1: Center-Filled Gummy Form

### Part A: Outer Gummy Shell Preparation

1. The pectin, sodium citrate, and approximately one third (⅓) of the sucrose according to the Formula in Table I are added as dry components to a plastic bag and manually mixed.
2. The citric acid is added to purified water and mixed to create a 50% solution according to the Formula in Table 1.
3. The water according to the Formula in Table 1 is heated to 90° C. in a suitable container.
4. The pectin, sodium citrate, and sucrose blend from Step 1 is added to the water from Step 3 and stirred manually until the pectin swells. The solution is brought to a boil.
5. The remaining sucrose (approximately two thirds (⅔) of the sucrose according to the Formula in Table 1) and citric acid/purified water mixture from Step 2 are added to the boiling mixture in Step 4 and mixed until dissolved.
6. The glucose syrup according to the Formula in Table 1 is placed into an additional container and brought to a boil, and then added to the mixture from Step 5 and mixed.
7. The ascorbic acid, elderberry extract, thyme, and marshmallow root according to the Formula in Table 1 are added to the mixture in Step 6 and mixed until dissolved.
8. The mixture in Step 7 is added to molds and allowed to cool to form a 4.0 g shell. The molds allow for a portion of a liquid center fill to be added in a separate step (Part B).

TABLE 1
Outer Gummy Shell Formula

| | Batch | Mg/ | % w/w for |
|---|---|---|---|
| Ingredient | Weight (g) | Shell | 4 g Shell |
| Pectin | 37.3 | 149.1 | 3.73 |
| Sodium Citrate | 2.8 | 11.2 | 0.28 |
| Sucrose | 301.3 | 1,205.0 | 30.13 |
| Glucose Syrup | 439.2 | 1,756.6 | 43.92 |
| Citric Acid | 9.3 | 37.3 | 0.93 |
| (as a 50% Solution) | | | |
| Ascorbic Acid | 17.0 | 68.0 | 1.70 |
| (as Vitamin C) | | | |
| Elderberry Extract | 43.8 | 175.0 | 4.38 |
| Thyme | 50.0 | 200.0 | 5.00 |
| Marshmallow Root | 6.0 | 25.0 | 0.60 |
| Water* | 265.6 (93.2) | 372.8 | 9.32 |
| TOTAL | 1,000 g | 4,000 mg | 100% w/w |

*A portion of water added during processing removed upon boiling. 172.4 g removed (93.2 g remains)

### Part B; Liquid Center Fill Preparation

1. The flavor and honey according to the Formula in Table 2 are mixed manually in a suitable container at room temperature until the flavor is dissolved.
2. The honey/flavor mixture from Step 1 is drawn into a sterile canula and injected into the center portion of the outer gummy shell prepared in Part A until 1.0 g of the honey/flavor mixture from Step 1 is deposited.

TABLE 2
Liquid Center Fill Formula

| | Batch | Mg/ | % w/w for 1 g |
|---|---|---|---|
| Ingredient | Weight (g) | Form | Center Fill |
| Honey | N/A | 999.5 | 99.95 |
| Flavor | | 0.5 | 0.05 |

1. A gummy form comprising a shell portion and a center fill portion, wherein the shell portion comprises marshmallow root.

2. The gummy form of claim 1, wherein the shell portion wholly encompasses the center fill portion.

3. The gummy form of claim 1, wherein the center fill portion comprises a liquid.

4. The gummy form of claim 3, wherein the center fill portion comprises honey.

5. The gummy form of claim 4, wherein the center fill portion comprises honey in an amount ranging from about 1 mL to about 4 mL.

6. The gummy form of claim 5, wherein the center fill portion comprises honey in an amount ranging from about 2 mL to about 3 mL.

7. The gummy form of claim 1, wherein the center fill portion comprises honey in an amount of about 2.5 mL.

8. The gummy form of claim 1, wherein the center fill portion is a solution, suspension, emulsion, or semisolid.

9. The gummy form of claim 1, wherein the center fill portion is substantially free of marshmallow root.

10. The gummy form of claim 1, wherein the shell portion comprises at least 51% of the total amount of marshmallow root.

11. The gummy form of claim 1, wherein the gummy form is substantially free of one or more of high fructose corn syrup, artificial sweeteners, artificial flavors, gluten, or gelatin.

12. The gummy form of claim 1, wherein the gummy form is substantially free of high fructose corn syrup, artificial sweeteners, artificial flavors, gluten, and gelatin.

13. The gummy form of claim 1, wherein the gummy form further comprises zinc, melatonin, vitamin C, elderberry, thyme, or combinations thereof.

14. The gummy form of claim 1, wherein the gummy form further comprises a sweetener selected from the group consisting of glycose syrup, stevia Rebaudioside A, Stevia leaf extract, monk fruit, sucrose, fructose, tapioca syrup, dextrose, and combinations thereof.

15. The gummy form of claim 1, wherein the gummy form is substantially free of an artificial sweetener.

16. The gummy form of claim 1, wherein the gummy form has a mass ranging from about 1 grams to about 8 grams.

17. The gummy form of claim 16, wherein the gummy form has a mass ranging from about 4 grams to about 6 grams.

18. The gummy form of claim 17, wherein the gummy form has a mass of about 5 grams.

19. A method of treatment of a subject suffering from a cough or upper respiratory condition by use of the gummy form according to claim 1.

20. The method of claim 19, wherein the subject is suffering from a cough, and the cough is due to hoarseness, dry throat, irritants, or a combination thereof.

21. The method of claim 19, wherein the subject is suffering from difficulty sleeping from the cough, and the gummy form comprises melatonin.
